(19) United States
(12) Patent Application Publication
(10) Pub. No.: US 2011/0224792 A1
(43) Pub. Date: Sep. 15, 2011

(54) ARTIFICIAL MUSCLE

(76) Inventor: Achim Groeger, Orionstr (DE)

(21) Appl. No.: 13/062,133

(22) PCT Filed: Apr. 15, 2009
(86) PCT No.: PCT/DE09/00519

§ 371 Date: May 23, 2011

(30) Foreign Application Priority Data
Sep. 3, 2008 (DE) 102008045554.7
Jan. 29, 2009 (DE) 202009001086.4

(51) Int. Cl. A61F 2/08

(52) U.S. Cl. 623/14.13

(57) ABSTRACT

The invention relates to an artificial muscle made of aplurality of nano-motors (referred to hereafter as nano power cells), wherein the nano-motors are the smallest unit for the production of complex muscular structures for generating longitudinal motor forces. The artificial muscle according to the invention is made of nano-motors (nano power cells), formed of symmetrical individual plates formed as double triangular segments and arranged radially in a honeycomb pattern, said plates being displaceable in the center and comprising an expansion unit in the interior thereof.

### BACKGROUND OF THE INVENTION

The invention relates to an artificial muscle that comprises a plurality of nano-motors (referred to hereinafter as nano-power cells), wherein the nano-motors are the smallest unit for producing complex muscular structures for generating longitudinal motor forces.

Complex structures such as for instance a muscle, the action of which is created from a plurality of serial and parallel nano-power cells, may be used for many different purposes such as for instance in prosthetics. Muscular structures whose properties are consistent with human and animal muscles can be produced based on the small nano-power cell. Lift and rotational movements are produced by the progress of the muscle structures.

In addition to employment in the field of prosthetics, the nano-power cell may be employed in all fields in which a longitudinal pulling force development can be used. This also applies to rotational movement sequences, the movement of which is produced from a plurality of longitudinal force machines.

Known from DE 36 44 481 A1 is a drive unit for movement mechanisms that may be employed with nothing further than as an implant in the field of biomedical engineering.

In this case the force generation element may be used as a muscle prosthesis. In the drive unit described, there is at least one force generation element that has an interior cavity closed by stiff end parts for limiting its working volume and of which one section can be securely connected to a first part of the mechanism and another section can be connected, in a tension-proof manner, to another part of the mechanism in order to effect a change in the relative position of the parts, and with a control unit for changing the working volume of the force generation element, wherein the force generation element has a radially elastic hose-like jacket, the longitudinal extension of which is limited by a support structure so that using the control device the inner surface-to-volume ratio may be changed with longitudinal change (ΔL) of the force generation element.

The longitudinal jacket is connected at its end to rigid plates, whose interval may be changed by raising or lowering the internal pressure so that either a shortening movement or a movement returning to the original length of the force generation element results. The individual elements are connected by a fluid connector to one end of the force generation element. Due to the smaller dimensions of the force generation elements, the fluid connectors must be embodied relatively thin, that is, like capillaries, so that a pressure can change only slowly, which, using the example of a muscle, leads to chameleon-like movement speeds.

Although muscle groups may be formed with round structures, they have disadvantages when performing rotational movements. Round structures for expanding bodies are not suited for rotational movements, for instance arm or foot rotation, because in the case of transversely arranged muscles, at the moment of force generation these structures pull into one another and thus the force required is consumed inside the muscle.

Generating the pressure represents another disadvantage. The units for generating the required pressures (pneumatic, hydraulic, etc.) must be embodied relatively large so that including these on a moving body is problematic.

Movement sequences, like for instance of the human skeleton, are fundamentally based on pulling movements. For using high-performance active elements (electroactive polymer (EAP) actuators, with varying characteristics or the nano-technology of the natural muscle), like of liquid crystals made of ferroelectric elastomers that permit rapid movement and are not sensitive to the environment, a design is necessary that converts a pressure action to a pulling action. For using high-performance nematic elastomers, with varying characteristics or the nano-technology of the natural muscle, like the nanotechnology of liquid crystals made of nematic elastomers, which permit rapid movements and are not sensitive to the environment, a design is necessary that produces a direct pulling action and that makes it possible to perform this action micromechanically in a non-positive fit.

### SUMMARY OF THE INVENTION

The technical problem addressed by the present invention is based on an extremely low-power drive with simultaneously high output power. The design of the nano-power cell drive is such that the latter is externally constructed such that the drive is tactilely very soft and visually is very similar to human and animal muscles.

The object and goal is a drive that is biologically similar to muscular structures and that has low power consumption for employment in a wide variety of technical and bionic fields.

The drive is formed from a plurality of nano-motors (nano-power cells).

The nano-power cell forms the smallest unit for producing complex muscular structures for generating longitudinal motor forces that convert a pressure action (polymers) into a pulling action or stabilize a contraction rubber (elastomers) such that the nano-power cell is stabilized in a non-positive fit as for the polymers.

Complex structures, such as for instance a muscle, the action of which is formed from serial and parallel nano-power cells, may be employed in diverse uses, such as for instance in prosthetics, internal medicine, robotics, engineering, etc. Muscular structures whose properties are consistent with human and animal muscles can be produced based on small nano-power cells. Lift and rotational movements are produced by the progress of the muscle structures.

The nano-power cell comprises a honeycomb-like jacketing in which is embedded the expansion unit that has been coiled in a spiral. The liquid crystal molecules are bound into polymer networks such that the latter exert a lifting action when an electrical field is applied. The expansion unit comprises a coil in which liquid crystals of ferroelectric elastomers are bound.

By applying an electrical field of 1.5 kV/mm to the liquid crystals, a volume expansion, currently approximately 4%, occurs in the nano-power cell. In practice the working voltage is in a range that is not harmful to humans or animals, that is, less than 25 volts.

By reducing the electrical field, the volume expansion returns to its original condition. The radial expansion that occurs in the center of the cell leads to the shortening of the nano-power cell, which is used as force for the movement sequences. Other variants from the EAP family may be used for the expansion unit, as well.

Another option for embodying the expansion unit is to design it as a contraction rubber comprising at least one nematic elastomer coil that is embodied with inlaid liquid crystals. The expansion unit embodied as a contraction rubber is embodied as a cylindrical hollow body that is provided all the way around with at least one nematic elastomer coil arranged along the surface of the hollow body.

Arranged inside the cylindrical hollow body is a heating device or a laser light source. It is connected to the mechanical couplings via an electrical connection.

The expansion unit comprises at least one nematic elastomer coil in which liquid crystals are bound and a heating device or laser light source is arranged in the interior of the elastomer coil. The nematic elastomer coils are heated or activated with the laser by applying a working voltage to the heating device or laser light source, which leads to a shortening of the nano-power cell and thus to a radial circumferential expansion in the center area.

In practice, the working voltage is in a range that is not harmful to humans or animals, that is, less than 25 volts. By decreasing the working voltage, the longitudinal change returns to its original status. The shortening of the nano-power cell leads to radial expansions that occur in the center of the nano-power cell and leads to the natural expansion of the muscle and at the same time to stabilization of rotating forces for the movement sequences.

The goal of the expansion unit is to reproduce the nano-motor muscles of living organisms like humans.

### BRIEF DESCRIPTION OF THE DRAWINGS

FIG. 1: is an exterior view of the nano-motor (nano-power cell);

FIG. 2: is a section through the nano-motor;

FIG. 3: is section A-A;

FIG. 4: is a sectional depiction of the expansion unit;

FIG. 5: is an expansion unit coiled in a spiral; and

FIG. 6: is a section through another embodiment of the nano-motor.

In a first exemplary embodiment, the inventive artificial muscle comprises the nano-motors 1 (nano power cells), which are formed from symmetrical individual plates 4 formed as double triangle segments and arranged radially in a honeycomb shape that are displaceable in the center and that have an expansion unit 5 inside. The outer ends are welded. Thus each nano-power cell 1 comprises six double triangle segments (individual plates 4) that are arranged in a honeycomb shape and that form the inner shell 2 and enclose the expansion unit 5 (FIG. 1).

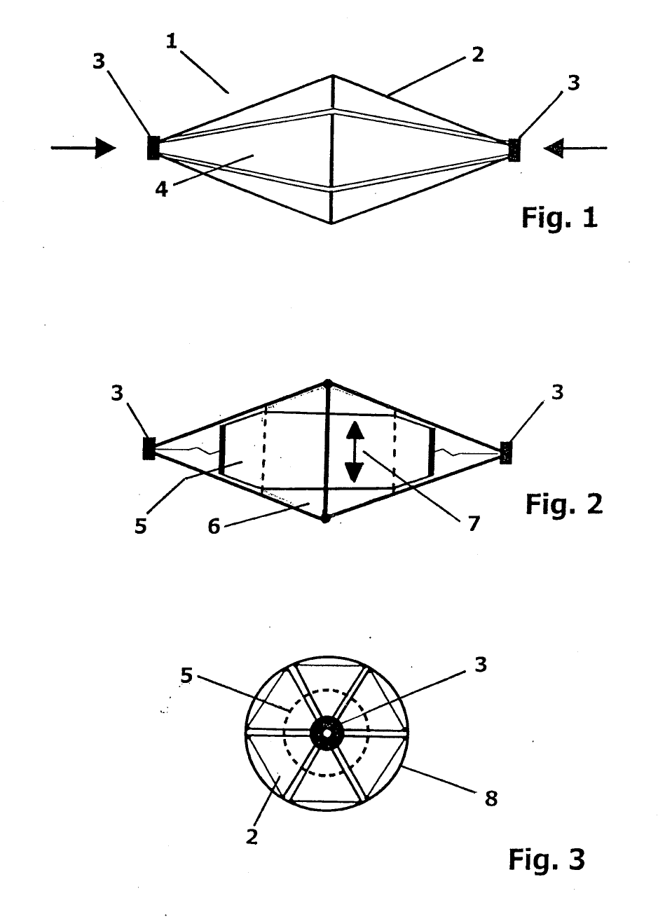

The intermediate space between the expansion unit 5 and the triangle segments 4 has a filling mass 6 that leads to the immediate change in the outer shell 8 when the expansion unit 5 expands (FIGS. 2 and 3).

The length of the nano-power cell 1 will be approximately four to six millimeters, depending on the application, for instance muscular structures for a prosthetic. The diameter depends on the number of coils for the plastic film that forms the expansion unit 5 and is between three and four millimeters when not expanded.

In order to return the nano-power cells 1 to their original state after expansion, they are enclosed by a slightly compressible material that simultaneously forms the insulator for the applied voltage.

Because of the volume expansion 7 of the expansion unit 5, the circumference in the center area of the nano-power cell 1 enlarges. Because of this expansion, the angle created causes pulling on the outer ends of the triangle segments 4, which leads to the nano-power cell 1 becoming shorter. This shortening produces the pulling force for the muscle. The pulling force is indispensable for human and animal skeletal structures to carry out movement sequences.

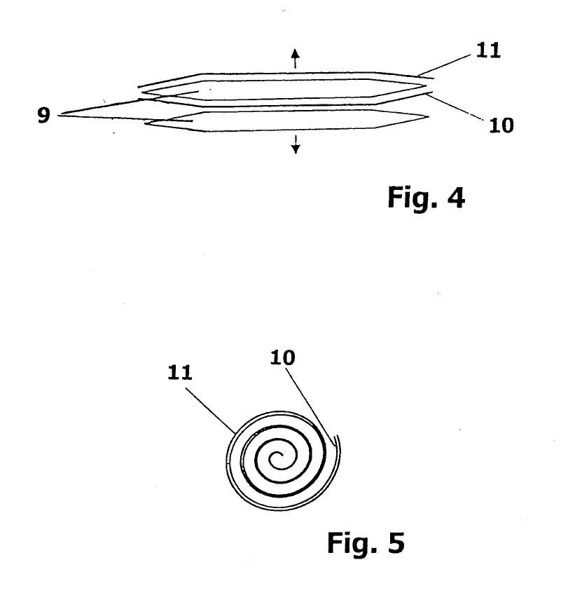

The expansion unit 5 (FIGS. 4 and 5) comprises two spiral-coiled plastic films that on both sides are conductive and easily expandable. The plastic films form the field plates (pole 1 and pole 2) 10, 11, between which the liquid crystals 9 are inlaid. The action of the expansion unit 5 is amplified because the liquid crystals 9 are inlaid in the spiral-coiled plastic film. This means that the expansion of the liquid crystals 9 takes place between the plastic films and between the coil.

The expansion of the liquid crystals 9 is produced by applying a voltage that permits an electrical field to act on the liquid crystals 9.

The liquid crystals 9 of the ferroelectric elastomers are rectified by the electrical field according to the field strength, which causes the volume expansion 7 by a lifting action of the molecules.

Reducing the field strength causes the liquid crystals 9 to return to an unordered state.

The reaction time of the expansion unit 5 can be measured in milliseconds; therefore a control unit that permits a uniform and adapted movement sequence must be employed.

The nano-power cell 1 is supplied with the control voltage for the electrical field via plug-in connectors/couplings 3 that are attached at both ends of the cell. The plug-in connectors 3 are connected to the expansion unit 5 via a flexible connection line.

The ends of the nano-power cells 1 are used as plug-in connectors that simultaneously act as a respective coupling 3 between the individual nano-power cells 1.

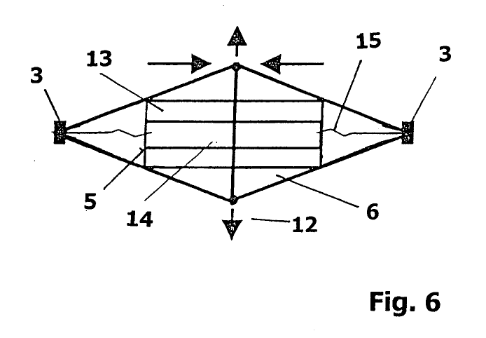

The intermediate space between the expansion unit 5 and the triangle segments 4 provides the space required for the radial expansion of the cylindrical rubber comprising nematic elastomers (FIG. 6).

The length of the nano-power cell 1 will be approximately four to six millimeters, depending on the application, for instance muscular structures for prosthetics.

In order to return the nano-power cells 1 to their original state after expansion, they are enclosed by a slightly compressible material that simultaneously forms the insulator for the applied voltage. In the case of the nematic elastomers, adding heat or light causes shortening of the nano-power cell 1. Because of its honeycomb-shaped structure, the radial circumference of the nano-power cell 1 is enlarged at the center.

This shortening produces the pulling force for the muscle. The pulling force is indispensable for human and animal skeletal structures to carry out movement sequences.

The expansion unit 5, as a contraction rubber, is embodied comprising a nematic elastomer coil 13 with inlays of liquid crystals. The expansion unit 5 has a structure comprising a cylindrical hollow body. The latter is provided all the way around with at least one nematic elastomer coil 13 running along the surface of the hollow body. A heating device or laser light source 14 is arranged inside the hollow body and is connected to the mechanical couplings 3 by means of the electrical connections. The hollow body and carrier of the elastomers is connected in a non-positive fit to the outer coupling elements.

The action of the expansion unit 5 is generated by inlaying the liquid crystals in the nematic elastomer coil 13.

By applying a voltage to the heating device or laser light source 14, the nematic elastomer coil 13 is heated or excited so that it draws into itself and a radial expansion 12 of the outer shell occurs. Reducing the voltage on the heating device or laser light source 14 causes the nematic elastomer coil 13 to return to its original state. The nano-power cell 1 is extended and thus the radial expansion 12 is reduced.

The reaction time for the expansion unit 5 is about 200 milliseconds, so a control unit that enables a uniform and adapted movement sequence must be employed.

The nano-power cell 1 is supplied with the working voltage for the heating device or the laser light source 14 via a plug-in connectors/couplings 3 that are attached to both ends of the cell. The plug-in connectors 3 are connected to the expansion unit 5 via a flexible electrical connector 15.

The ends of the nano-power cells 1 are used as plug-in connectors that simultaneously act as a coupling 3 between the individual nano-power cells 1.

A plurality of nano-power cells 1 to create complex muscle packets may be arranged as desired in any geometric shape. One must always proceed from the fact that a muscle packet can only pull. Each counteraction must be undertaken by a complementary muscle packet.

When muscle packets are in complex geometric formations, the control mechanism for all cooperating muscle packets must be stepped in a correspondingly fine manner. Very small cell structures are created by employing the nano-motor technology. When a voltage is applied, the nano-motors 1 cause a volume expansion 7. This causes an expansion of the muscle cell, which causes a shortening of the muscle cell (nano-power cell 1).

In another embodiment, a very small cell structure is created by employing the nano-motor technology. When a voltage is applied, the nano-motors 1 cause a contraction of the elastomer rubber (elastomer coil 13). This causes a shortening of the muscle cell, so that a radial enlargement of the muscle cell (nano-power cell 1) occurs.

The expansion acts on the six rhombic individual plates 4 that are arranged in a honeycomb shape. It is not possible to perceive a significant difference between the combination of the parallel and serial arrangement of the nano-power cells 1, purely exteriorly, and natural muscles. In terms of tactile perception, it is possible to come very close to the consistencies of human muscles.

The energy demand for the artificial muscle, which comprises a plurality of nano-power cells 1, is approx. 500 watts at peak power. This power in needed in order to produce the normal forces e.g. for a person. A fuel cell unit, which can be obtained in very small sizes, is used. It is possible to house this fuel cell in the interior of the artificial bone. The heat that develops from the fuel cell is used for heating the artificial muscles to body temperature.

Longitudinal forces are created immediately for all necessary lifting actions. The honeycomb structure ensures high mechanical efficiency.

Rotations can be created by the honeycomb-shaped outer surface since when this happens there is necessarily mutual stabilization of the individual elements of the artificial muscle and thus large rotational forces may be attained.

High pulling forces may be realized by dividing six rhombic pulling elements in one honeycomb structure, especially by the ductile behavior of the individual self-stabilizing rhombic elements.

Rapid movement sequences may be attained in about 100 milliseconds, which would be very fast for everyday activities. All movement sequences are controlled so that both slow and rapid movements are possible.

The control elements for triggering the cells are purely electronic. There are no mechanical components required for control, such as for instance valves. All movement sequences are silent. The voltages used are less than 25 volts and are not harmful for living organisms like humans and animals.

The processor-controlled computer units that are networked to one another are maintained in the interior of the large bones.

The software controls continuous movement sequences with self-learning algorithms.

The application field of the present invention extends to all technical applications in which mechanical force-controlled movement sequences are required.

The drive of the present invention is significant for bionic fields of use in order to produce the latest generation of prosthetics. The advantage of this drive is its great similarity to natural muscular drives for human and animal body functions. by means of complex control mechanisms, sensor systems comprising hardware and software, linking to the nervous system, drive energy from hydrogen fuel cells, and the most modern connections of natural bone parts to artificial materials for artificial bone construction.

The fields of application for the artificial muscle are quite diverse. It may be employed in prosthetics, internal medicine, robotics, and in general technical applications with forces that pull longitudinally.

1. An artificial muscle, comprising:
at least one nano-motor being formed from an expansible outer shell comprising stable longitudinal structure; and
an expansion unit arranged within an interior of the outer shell.

2. An artificial muscle in accordance with claim 1, wherein
the outer shell comprises six rhombic individual plates that are affixed to one another by flexible connections.

3. An artificial muscle in accordance with claim 1, wherein
the expansion unit comprises a film comprising inlays of liquid crystals of ferromagnetic elastomers comprising electroactive polymers.

4. An artificial muscle in accordance with claim 3, wherein
the film is coiled radially.

5. An artificial muscle in accordance with claim 4, wherein
the film comprises at least three layers, wherein outer layers comprise a non-conducting material, the surface of which is coated on both sides with an electrically conducting material, and an intermediate layer comprises the liquid crystals of charged ferromagnetic elastomers.

6. An artificial muscle in accordance with claims 1, wherein
the expansion unit comprises a contraction rubber including at least one nematic elastomer coil comprising inlays of liquid crystals.

7. An artificial muscle in accordance with claim 6, wherein
the expansion unit is configured as a cylindrical hollow body and is provided all the way around with the at least one nematic elastomer coil being arranged along the surface of the hollow body.

8. An artificial muscle in accordance with claim 7, wherein
arranged inside the expansion unit that is configured as a cylindrical hollow body is a heating device or a laser light source, and each of the expansion unit and heating device or laser light source is connected to mechanical couplings positioned at ends of the nano-motor outer shell via an electrical connection.

9. An artificial muscle in accordance with claim 2, wherein
integrated on the end faces of the outer shell are mechanical couplings comprising electrical plug-in connectors serving to hold end area portions of the rhombic individual plates.

10. An artificial muscle in accordance with at least one of the foregoing claims, wherein
the outer shell is provided with a spring-loaded covering.

11. Artificial muscle in accordance with claim 10, wherein
the spring-loaded covering comprises a non-conducting plastic functioning as an insulator for the nano-motor and promotes mechanical compression.

12. An artificial muscle in accordance with claim 1, wherein
a space between the expansion unit and the inside of the outer shell is filled with a flexible plastic.
